(19) United States
(12) Patent Application Publication
(10) Pub. No.: US 2005/0215819 A1
(43) Pub. Date: Sep. 29, 2005

(54) Method of producing an amide

(76) Inventor: Lawrence Joseph Williams, Edison, NJ (US)

Correspondence Address: Jane Massey Licata;Licata & Tyrrell P.C., 66 E. Main Street, Marlton, NJ, 08053, US

(21) Appl. No.: 10/807,206

(22) Filed: Mar. 23, 2004

(51) Int. Cl. C07C 231/10

(52) U.S. Cl. 564/133

(57) ABSTRACT

The present invention discloses a new method for synthesizing an amide based on a fundamental mechanistic revision of the reaction of thio acids and organic azides. Moreover, the application of this method to the selective preparation of several classes of complex amides in nonpolar and polar solvents, including water, is provided.

### BACKGROUND OF THE INVENTION

Conventional methods for the chemical synthesis of amides utilize active esters and amines as precursors and are efficient at producing simple peptide products. Nevertheless, many classes of amides, including those found in many natural products, bioconjugates, and pharmaceutical candidates, pose significant challenges to these methodologies.

### SUMMARY OF THE INVENTION

The present invention is a method of producing an amide by combining a thio acid and an organic azide in the presence of a solvent.

### DETAILED DESCRIPTION OF THE INVENTION

Conventional methods of producing amides utilize thioacetic acid, applied as solvent or cosolvent, acting upon organic azides to provide the corresponding acetamide product (eq 1) (Rosen, et al. (1988) J. Org. Chem. 53:1580; Rakotomanomana, et al. (1990) Carbohydr. Res. 197:318; Hakimelahi and Just (1980) Tetrahedron Lett. 21:2119).

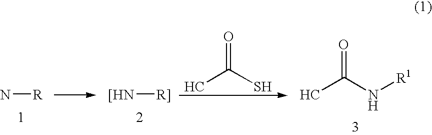

A reaction mechanism wherein the azide is reduced in situ to give the corresponding amine (2) followed by unusually rapid acetylation of the amine intermediate has been proposed (Rosen, et al. (1988) supra). It was suggested that thioacetic acid-induced formation of amides from azides involves a very rapid, but otherwise conventional, nucleophilic acyl substitution reaction.

As used herein, R and R¹ substituents comprise ethyl, methyl, phenyl, propyl, isopropyl, butyl, isobutyl, carbonyl, methoxy, ethoxy, n-propoxy, as well as any other simple or complex organic molecule.

It was determined whether a free amine is an obligatory intermediate. Treatment of benzylamine in dichloromethane (0.5 M) with trifluoroacetic acid (1.0 equiv) followed by a slight excess (1.3 equiv) of thioacetic acid gave virtually no amide product (<4%) after 15 hours at room temperature. Benzyl azide under these conditions, however, gave N-benzyl acetamide in 42% yield. Benzenesulfonyl azide reacted in minutes upon exposure to thio acids (e.g., thioacetic acid or thiobenzoic acid) to form N-acyl sulfonamides in excellent yields (>95%), whereas benzenesulfonamide failed to react even after several days. These results indicate that thio acids react with organic azides to give amide products without prior reduction to the amine.

Other organic azides bearing electron-withdrawing functionality (Table 1) were examined in the synthesis of an amide. N-acyl carbamates (entry 2), N-aryl amides (entry 3), and, unexpectedly, enamides (entry 4) were efficiently prepared under very mild conditions.

TABLE 1

| Entry | Azide | ° C./Time/Solvent | Amide | Yield |
|---|---|---|---|---|
| 1 | 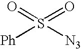 | a) 25/15 minutes/MeOH b) 25/15 minutes/MeOH | 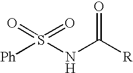 | a) 98% b) 96% |
| 2 | 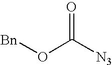 | a) 25/2 hour/MeOH b) 25/2 hour/MeOH | 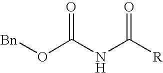 | a) 99% b) 96% |
| 3 | 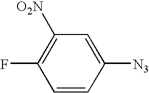 | a) 25/15 hour/MeOH b) 25/15 hour/MeOH | 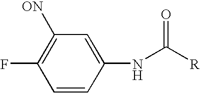 | a) 95% b) 94% |
| 4 | 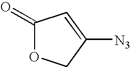 | a) 0/2 hour/MeOH b) 0/2 hour/MeOH | 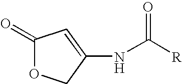 | a) 98% b) 95% |

^{a} Conditions: 0.94-0.024 M azide; 1:1.3:1.3 azide:2,6-lutidine:thio acid. (a) Thiobenzoic acid, R = C₆H₅. (b) Thioacetic acid, R = CH₃.

Similarly, electron-rich azides coupled with thio acids (Table 2); however, heating and base additives were found to be necessary for more challenging alkyl substrates (entries 2-5). E/Z mixtures of β-azido styrene provided exclusively the (E)-enamide products (entry 3). In contrast, when exposed to thio acid, the unprotected hydroxy azide (entry 4) was selectively converted to the hydroxy amides without measurable side reaction or epimerization of the azide.

TABLE 2

| Entry | Azide | ° C./Time/Solvent | Amide | Yield |
|---|---|---|---|---|
| 1 | 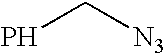 | a) 60/15 hour/CHCl₃b) 60/15 hour/ CHCl₃ | 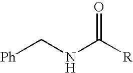 | a) 78% b) 86% |
| 2 | 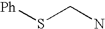 | a) 60/15 hour/CHCl₃b) 60/15 hour/ CHCl₃ | 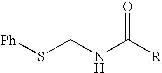 | a) 77% b) 85% |
| 3 | 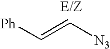 | a) 60/10 hour/CHCl₃b) 60/18 hour/ CHCl₃ | 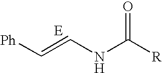 | a) 66% b) 79% |
| 4 | 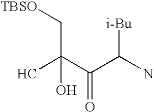 | a) 25/30 hour/CHCl₃b) 60/24 hour/ CHCl₃ | 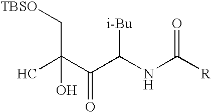 | a) 94% b) 88% |
| 5 | 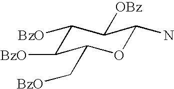 | a) 60/36 hour/CHCl₃b) 60/36 hour/ CHCl₃ | 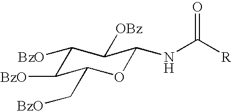 | a) 64% b) 97% |

^{a} Conditions: 1.0-0.18 M azide; 1:1.3-2.5:1.3-2.6 azide:2,6-lutidine:thio acid. (a) Thiobenzoic acid, R = C₆H₅. (b) Thioacetic acid, R = CH₃. For entry 5a, yield based on recovered starting material: 95%.

The direct conversion of glycosyl azides to the N-acyl products was also examined. It should be noted that glycosylamines are configurationally unstable under many acylation reaction conditions (Cohen-Anisfeld and Landsbury (1993) J. Am. Chem. Soc. 115:10531; Tamura, et al. (1984) Bull. Chem. Soc. Jpn. 57:3167; Damkaci and DeShong (2003) J. Am. Chem. Soc. 125), whereas glycosyl azides are configurationally stable. Conversions took place in good yield (entry 5, Table 2), and the reactions proceeded with complete stereochemical fidelity.

The synthesis of amides in water (Table 3) was also examined. β-Glucosyl azide was cleanly converted to the β-N-amidoglycoside without isomerization (entry 1) (Tamura, et al. (1984) supra; Damkaci and DeShong (2003) supra), 3′-azido-3′-deoxythymidine was converted to the corresponding amides (entry 2), and N-acyl sulfonamides (entry 3) were produced without complication in aqueous solution.

TABLE 3

| Entry | Azide | ° C./Time/Solvent | Amide | Yield |
|---|---|---|---|---|
| 1 | 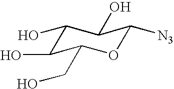 | a) 60/36 hours/H₂O b) 60/36 hours/H₂O | 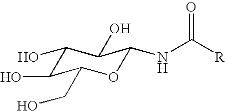 | a) 83% b) 80% |
| 2 | 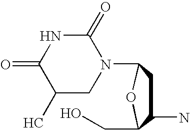 | a) 60/36 hours/H₂O b) 60/36 hours/H₂O | 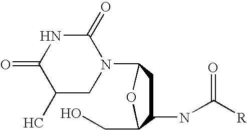 | a) 68% b) 77% |
| 3 | 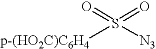 | a) 25/1 hour/H₂O b) 25/1 hour/H₂O | 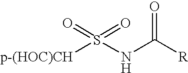 | a) 93% b) 98% |

^{a} Conditions: 0.25-0.040 M azide; 1:1.3-5 azide:thio acid; entry 1, NaHCO₃(aq); entry 2, PBS buffer pH 7.4; entry 3, 1.8 equiv of 2,6-lutidine. (a) Thiobenzoic acid, R = C₆H₅. (b) Thioacetic acid, R = CH₃.

The entries in Table 4 illustrate the preparation of N-acetyl R-amino acyl sulfonamides from thioesters 8a-c. Liberation of the thio acid, followed by treatment with sulfonyl azide, gave 9a-e. Hence, sophisticated thio acids participate in this reaction as well. No epimerization of the thio acid partner occurred as determined by careful comparison of the diastereomeric products from entries 2 and 3. Entries 1-3 also demonstrate a new route to highly useful “safety catch” linkers (Backes and Ellman (1999) J. Org. Chem. 64:2322), while entries 4 and 5 represent C-terminal fluorescently labeled peptide derivatives.

TABLE 4

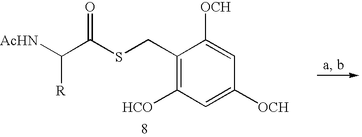

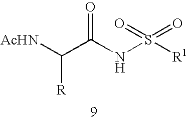

| | | | | | Yield |
|---|---|---|---|---|---|
| Entry | 8 | R | Azide | 9 | (two steps) |
| 1 | a | i-Bu | N₃-Bs | 9a, N-Ac-Leu-NH-Bs | 91% |
| 2 | b | (R)-sec-Bu | N₃-Ts | 9b, N-Ac-alle-NH-Ts | 87% |
| 3 | c | (S)-sec-Bu | N₃-Ts | 9c, N-Ac-Ile-NH-Ts | 72% |
| 4 | b | (R)-sec-Bu | N₃₋ | 9d, | 73% |
| | | | dansyl | N-Ac-alle-NH-dansyl | |
| 5 | a | i-Bu | N₃₋ | 9e, | 73% |
| | | | dansyl | N-Ac-Leu-NH-dansyl | |

^{a} Conditions: (a) TFA/DCM (40-80% v/v), HsiEt₃; (b) CH₃OH, 0.16-0.17 M thio acid; 2-5 equiv of azide; 3-6 equiv of 2,6 lutidine, room temperature.

Equation 2 presents a new mechanistic framework for the synthesis method provided herein. Formation of a thiatriazoline intermediate (6), rather than reduction of the azide to amine, accounts for the observations provided herein and in other studies (Rosen, et al. (1988) supra; Rakotomanomana, et al. (1990) supra; Hakimelahi and Just (1980) supra; Marcaurelle and Bertozzi (2001) J. Am. Chem. Soc. 123:1587; Elofsson, et al. (1997) Tetrahedron 53:369; Chou, et al. (1997) J. Chem. Soc., Perkin Trans. 1:1691; McKervey, et al. (1993) J. Chem. Soc. Chem. Commun. 94; Paulsen, et al. (1994) Liebigs Ann. Chem. 369). This intermediate may form via either a 2+3 cycloaddition or a stepwise diazo transfer-like mechanism. Decomposition of 6, stepwise or by a retro-[2+3] reaction, would ultimately lead to amide, nitrogen, and sulfur (Loock, et al. (1973) J. Org. Chem. 38:2916; L'abbe, et al. (1975) J. Org. Chem. 40:1728; L'abbe, et al. (1990) J. Heterocycl. Chem. 27:1059; L'abbe (198) Angew. Chem., Int. Ed. Engl. 19:276).

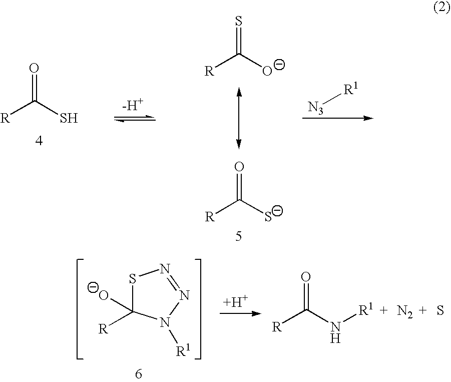

Thio acid/azide coupling has several advantages over conventional amidation reactions. Amine analogues of azides in Tables 1 and 4 would resist mild acylation conditions due to significantly reduced nucleophilic properties, whereas amine analogues of Table 2, entries 2-5, would be expected to undergo facile side reactions. In addition, many problems in amide synthesis are exacerbated in methanol and water, where amine nucleophilicity is reduced, and active esters are rendered susceptible to solvolysis (see Tables 1, 3, and 4). Thus, using the method of the invention, both simple and complex amides difficult to access using conventional methods have been prepared without the use of protecting groups and in aqueous solution.

Accordingly, the present invention is a method of producing an amide by combining a thio acid and organic azide in the presence of a solvent. In one embodiment, the thio acid is not used as a solvent or cosolvent. In another embodiment, the conversion of azides to amides does not involve the reduction of the azide to the corresponding amine.

Organic azides which may be used in accordance with the method of the invention include compounds having the azide group attached directly or indirectly, by nonionic bonding, to a carbon of an organic compound, wherein the azide group has no single definite structure; it can be represented by different resonance forms. Examples of suitable organic azide compounds include, but are not limited to, those exemplified herein, 4-azidobenzoic acid, 4-acetamidobenzenesulfonyl azide, azidoacetic acid ethyl ester, D(−)-α-azido-α-phenylacetyl chloride, diphenylphosphoryl azide, trimethylsilyl azide, 4-toluenesulfonyl azide, and the like.

A thio acid is considered an organic compound produced by replacement of one of the oxygens of a carboxyl group by divalent sulfur. Examples of suitable thio acid compounds for use in the method of the present invention include, but are not limited to, those exemplified herein, thioglycolic acid, thiodiglycolic acid, thio salicylic acid, and the like.

Organic azides and thio acids are combined in a ratio of 0.2-1.0 azide to 1.0-5.0 thio acid, or 0.5-1.0 azide to 1.0-2.6 thio acid, or 0.75-1.0 azide to 1.0-1.3 thio acid.

A reaction solvent may be protic, aprotic, polar or nonpolar and includes, but is not limited to, methanol, chloroform, water, and other hydroxylic solvents. Further, a solvent such as 2,6-lutidine is useful as it was found to significantly accelerate the reaction and was superior to other bases, including pyridine and 2,6-di-tert-butyl pyridine. It has been found that yields depend primarily upon the electronic and steric properties of the azide and secondarily upon the thio acid. The solvent is combined with the thio acid and azide at a ratio of 1:1.0-6.0, or 1:1.0-3.0, or 1:1:3-2.0 azide:solvent.

A reaction of the invention can be carried out at a temperature between −78° C. and 250° C., or can be carried out between 0° C. and 100° C., or between 10° C. and 60° C., with or without agitation for a sufficient amount of time (e.g., 15 minutes, 1 hour, 2 hours, 10 hours, 30 hours, 50 hours or more) to produce a suitable yield (e.g., 50%, 60%, 75%, 85%, 95% or more). The resulting product can be analyzed using standard methodologies such as TLC, HPLC, NMR, high resolution MS, MS-MS, elemental analysis, IR and the like to determine purity and structure.

Tables 1-4 summarize exemplary simple and complex amide products which can be formed in accordance with the method of the invention thereby avoiding the use of thio acid as solvent or cosolvent.

Amides produced by the method of the invention can contain pure enantiomers or pure diastereomers or mixtures of enantiomers, for example in the form of racemates, or mixtures of diastereomers. Mixtures of two or more stereoisomers are further contemplated with varying ratios of stereoisomers in the mixtures. Amides can also contain trans- or cis-isomers including pure cis-isomers, pure trans-isomers or cis/trans-isomer mixtures with varying ratios of each isomer. When a composition containing a pure compound is desired, diastereomers (e.g., cis/trans-isomers) can be separated into the individual isomers (e.g, by chromatography) or racemates (e.g., separated using standard methods such as chromatography on chiral phases or resolution by crystallization of diastereomeric salts obtained with optically active acids or bases). Stereochemically uniform amides can also be obtained by employing stereochemically uniform reactants or by using stereoselective reactions.

In the syntheses, purification and identification of the compounds produced in accordance with the method of the present invention, the compounds can be present in free and salt form, therefore as used herein, a free compound should be understood as including the corresponding salts.

It is contemplated that the method of the invention will be useful in conjunction with recent advances in protein synthesis (Tam, et al. (2001) Biopolymers 60:194; Offer and Dawson (2000) Org. Lett. 2:23; Offer, et al. (2002) J. Am. Chem. Soc. 124:4642), engineering (Cornish, et al. (1995) Angew. Chem., Int. Ed. Engl. 34:621; Chin, et al. (2002) J. Am. Chem. Soc. 124:9026; Beligere and Dawson (2000) J. Am. Chem. Soc. 122:12079), as well as unconventional amide synthesis approaches (Damkaci and DeShong (2003) supra; Saxon and Bertozzi (2000) Science 287:2007; Saxon, et al. (2000) Org. Lett. 2:2141; Nilsson, et al. (2000) Org. Lett. 2:1939; Nilsson, et al. (2001) Org. Lett. 3:9; Humphrey and Chamberlin (1997) Chem. Rev. 97:2243; Park, et al. (2002) Tetrahedron Lett. 43:6309; Suh and Kishi (1994) J. Am. Chem. Soc. 116:11205). Considering the ease of preparation of azides and thio acids in solution and on solid support (Scriven and Turnbull (1988) Chem. Rev. 88:297; Rijkers, et al. (2002) Tetrahedron Lett. 43:3657; Goldstein and Gelb (2000) Tetrahedron Lett. 41:2797; Rajagopalan, et al. (1997) Synth. Commun. 27:187; Canne, et al. (1995) Tetrahedron Lett. 36:1217; Schwabacher and Maynard (1993) Tetrahedron Lett. 34:1269), the method of the present invention will be useful in the construction of natural and designed peptides and amide-containing natural products. Further, it is contemplated that sophisticated plastics, biopolymers, composite materials, molecular tools for cell biology, and medicinal agents (e.g., zampanolide or epoxomicin) can also be produced.

The invention is described in greater detail by the following non-limiting examples.

### EXAMPLE 1

### General

Starting materials, reagents and solvents were purchased from commercial suppliers (SIGMA-Aldrich, St. Louis, Mo.; Bachem AG, Bubendorf, Switzerland; Advanced Chem Tech, Louisville Ky.; Fischer, Fairlawn, N.J.) and used without further purification. All reactions were conducted in oven-dried (135° C.) glassware under an inert atmosphere of dry nitrogen. The progress of reactions was monitored by Silica gel thin layer chromatography (TLC) plates (mesh size 60 Å with fluorescent indicator, SIGMA-Aldrich), visualized under UV and charred using cerium or anisaldehyde stain. Products were purified by flash column chromatography (FCC) on 120-400 mesh silica gel (Fisher). Melting points were recorded on a Thomas Hoover capillary melting point apparatus and are uncorrected. Infrared (FTIR) spectra were recorded on an ATI Mattson Genesis Series FT-Infrared spectrophotometer. Proton nuclear magnetic resonance spectra (¹H NMR) were recorded on either a Varian-300 instrument (300 MHz), or a Varian-400 instrument (400 MHz). Chemical shifts are reported in ppm relative to tetramethylsilane (TMS) as the internal standard. Data is reported as follows: chemical shift, integration, multiplicity (s=singlet, d=doublet, t=triplet, q=quartet, br=broad, m=multiplet), and coupling constants (Hz). Carbon nuclear magnetic resonance spectra (¹³C NMR) were recorded on either a Varian-300 instrument (75 MHz), or a Varian-400 instrument (100 MHz). Chemical shifts are reported in ppm relative to tetramethylsilane (TMS) as the internal standard. Mass spectra were recorded on a Finnigan LCQ-DUO mass spectrometer.

### EXAMPLE 2

### General Procedure for Preparing Amides from Azides

RN₃+R′COSH→RNHCOR′

To a stirred solution of azide in methanol (or DCM/CHCl₃, water) was added 2,6-lutidine followed by drop-wise addition of thioacid under inert atmosphere. The reaction mixture was stirred and monitored by TLC. After completion of the reaction, the solvent was evaporated and the residue was dried under vacuum. The product was normally purified by a silica gel flash column chromatography (FCC), using ethyl acetate/acetone-hexane as the eluent.

### EXAMPLE 3

### Synthesis of Exemplary Amides

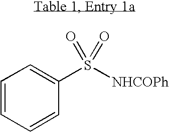

The reaction for the synthesis of entry 1a (Sturino and Labelle (1998) Tetrahedron Lett. 39: 5891-5894) was carried out following the general procedure, using 126 mg (0.689 mmol) of azide, 96 mg (0.897 mmol) of 2,6-lutidine and 124 mg (0.899 mmol) of thiobenzoic acid in methanol (conc. of azide 0.7 M) at room temperature for 15 minutes. FCC (silica gel, 33% ethyl acetate-hexane) gave 177 mg (98%) of 1a as a white solid; mp: 148-149° C. (ref: 146-147° C.); IR νₘₐₓ (neat)/cm⁻¹ 3282, 3062, 1718; δ_{H} (300 MHz, Acetone-d₆) 11.10-10.90 (1H, bs, NH), 8.13 (2H, d, J=6.9 Hz, ArH), 7.93 (2H, d, J=7.2 Hz, ArH), 7.75-7.60 (4H, m, ArH), 7.50 (2H, t, J=8.1 Hz, ArH); δ_{C} (75 MHz, Acetone-d₆) 165.4, 140.4, 134.3, 133.8, 132.5, 129.5 (2), 129.3 (2), 128.9 (2), 128.8 (2); m/z (ESIMS) 262 (M+1)⁺.

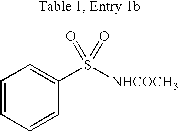

The reaction for the synthesis of entry 1b (Hermann, et al. (1992) J. Org. Chem. 57: 5328-5334) was carried out following the general procedure, using 124 mg (0.678 mmol) of azide, 94 mg (0.879 mmol) of 2,6-lutidine and 67 mg (0.882 mmol) of thioacetic acid in methanol (conc. of azide 0.7 M) at room temperature for 15 minutes. FCC (silica gel, 33% acetone-hexane) gave 130 mg (96%) of 1b as a white solid; mp: 121-124° C. (ref: 124.5-126° C.); IR νₘₐₓ (neat)/cm⁻¹ 3121, 2902, 1688; δ_{H} (400 MHz, CDCl₃) 9.20-8.90 (1H, bs, NH), 8.07 (2H, d, J=8.0 Hz, ArH), 7.67 (1H, t, J=7.2 Hz, ArH), 7.57 (2H, t, J=8.0 Hz, ArH), 2.08 (3H, s, CH₃); δ_{C} (100 MHz, CDCl₃) 168.4, 138.4, 134.1, 129.1 (2), 128.3 (2), 23.5; m/z (ESIMS) 198 (M−1)⁻.

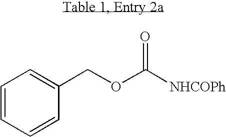

The reaction for the synthesis of entry 2a (Bailey, et al. (2001) J. Chem. Soc. Perkin Trans. 1, 3245-3251) was carried out following the general procedure, using 100 mg (0.565 mmol) of azide, 78 mg (0.730 mmol) of 2,6-lutidine and 101 mg (0.732 mmol) of thiobenzoic acid in DCM (conc. of azide, 0.94 M) at room temperature for 15 hours. FCC (silica gel, 1:3 ethyl acetate-hexane) gave 142 mg (99%) of 2a as a white solid. mp: 111-113° C. (ref: 112-114° C.); IR νₘₐₓ (neat)/cm⁻¹ 3296, 1763; δ_{H} (400 MHz, CDCl₃) 8.28 (1H, bs, NH), 7.81 (2H, d, J=7.6 Hz, ArH), 7.57 (1H, t, J=7.2 Hz, ArH), 7.46 (2H, t, J=7.6 Hz, ArH), 7.42-7.30 (5H, m, ArH), 5.24 (2H, s, CH₂); δ_{C} (100 MHz, CDCl₃) 164.9, 150.9, 135.0, 133.0 (2), 132.9, 128.9 (2), 128.71 (2), 128.69 (2), 127.6 (2), 68.0; m/z (ESIMS) 278 (M+Na)⁺.

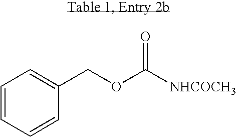

The reaction for the synthesis of entry 2b (Lucente, et al. (1978) Tetrahedron Lett. 3155-3158) was carried out following the general procedure, using 51.6 mg (0.292 mmol) of azide, 41 mg (0.383 mmol) of 2,6-lutidine and 29 mg (0.382 mmol) of thioacetic acid in methanol (0.71 M conc. of azide) at room temperature for 15 hours. FCC (silica gel, 30% ethyl acetate-hexane) gave 54.2 mg (96%) of 2b as a white solid. mp: 104-105° C. (ref: 104° C.); IR νₘₐₓ (neat)/cm⁻¹ 3209, 3143, 2921, 1747, 1687; δ_{H} (400 MHz, CDCl₃) 7.70 (1H, bs, NH), 7.41-7.34 (5H, bs, ArH), 5.18 (2H, s, CH₂), 2.43 (3H, s, CH₃); δ_{C} (100 MHz, CDCl₃) 172.0, 151.8, 134.9, 128.8, 128.7 (2), 128.4 (2), 67.9, 24.0; m/z (ESIMS) 216 (M+23)⁺.

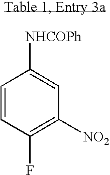

The reaction was carried out following the general procedure, using 40 mg (0.220 mmol) of azide, 30 mg (0.283 mmol) of 2,6-lutidine and 40 mg (0.288 mmol) of thiobenzoic acid in methanol (0.44 M conc. of azide) at room temperature for 15 hours. The reaction mixture was concentrated to dryness and the crude product was washed with hexane and dried under vacuum to furnish 54 mg of 3a (95%) as a yellow solid. mp: 174-177° C.; IR νₘₐₓ (neat)/cm⁻¹ 3312, 1652, 1532; δ_{H} (300 MHz, Acetone-d₆) 9.96 (1H, bs, NH), 8.78 (1H, dd, J=6.9, 2.7 Hz, ArH), 8.25-8.19 (1H, m, ArH), 8.02-8.06 (2H, m, ArH), 7.47-7.67 (4H, m, ArH); δ_{C} (75 MHz, Acetone-d₆) 166.5, 153.6, 150.1, 137.0, 135.2, 132.8 (2), 129.3 (2), 128.3 (2), 127.8, 119.2, 117.5; m/z (ESIMS) 259 (M−1)⁻.

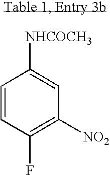

The reaction for the synthesis of 3b (McFarlane, et al. (1988) J. Chem. Soc., Perkin Trans. 1, 691-696) was carried out following the general procedure, using 33 mg (0.181 mmol) of azide, 25 mg (0.232 mmol) of 2,6-lutidine and 18 mg (0.238 mmol) of thioacetic acid in methanol (0.45 M conc. of azide) at room temperature for 15 hours. The reaction mixture was concentrated to dryness and the crude product was washed with hexane and dried under vacuum to furnish 34 mg (94%) of 3b as a yellow solid. mp: 137-139° C. (ref 140-141° C.); IR νₘₐₓ (neat)/cm⁻¹ 3355, 1672, 1534; δ_{H} (300 MHz, Acetone-d₆) 9.60 (1H, bs, NH), 8.57 (1H, dd, J=6.9, 2.7 Hz, ArH), 7.94-7.88 (1H, m, ArH), 7.43 (1H, dd, J=11.1, 9.0 Hz, ArH), 2.13 (3H, s, CH₃); δ_{C} (75 MHz, Acetone-d₆) 168.8, 152.6, 149.2, 136.4, 126.1, 118.6, 115.7, 23.7; m/z (ESIMS) 140 (M−1)⁻.

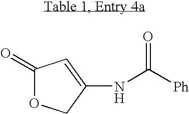

The reaction was carried out following the general procedure, using 88 mg (0.704 mmol) of azide, 97 mg (0.905 mmol) of 2,6-lutidine and 129 mg (0.933 mmol) of thiobenzoic acid in methanol (0.44 M conc. of azide) at room temperature for 2 hours. The crude product was washed with dichloromethane and acetone to furnish 140 mg (98%) of 4a as a white solid. mp: 242-244° C. (decompose); IR νₘₐₓ (neat)/cm⁻¹ 3287, 1711, 1607; δ_{H} (300 MHz, DMSO-d₆) 10.20 (1H, bs, NH), 6.87 (2H, bs, ArH)), 6.56 (1H, bs, ArH), 6.47 (2H, bs, ArH), 4.83 (1H, bs, CH), 4.04 (2H, bs, CH₂); δ_{C} (100 MHz, DMSO-d₆) 173.8, 165.8, 161.0, 133.0, 132.4, 128.8 (2), 128.0 (2), 96.1, 69.3; m/z (ESIMS) 202 (M−1)⁻.

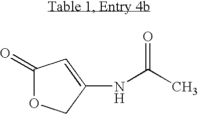

The reaction was carried out following the general procedure, using 96 mg (0.768 mmol) of azide, 107 mg (1 mmol) of 2,6-lutidine and 76 mg (1 mmol) of thioacetic acid in methanol (0.48 M conc. of azide) at room temperature for 2 hours. The crude product was washed with dichloromethane and hexane to furnish 103 mg (95%) of 4b as a white solid. mp: 198-200° C.; IR νₘₐₓ (neat)/cm⁻¹ 3200, 3030, 1715; δ_{H} (400 MHz, Acetone-d₆) 10.20 (1H, bs, NH), 5.63 (1H, s, CH), 5.02 (2H, d, J=1.2 Hz, CH₂), 2.14 (3H, s, CH₃); δ_{C} (100 MHz, Acetone-d₆) 174.0, 169.8, 161.0, 95.8, 69.7, 23.6; m/z (ESIMS) 140 (M−1)⁻.

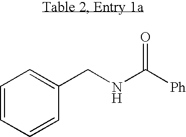

The reaction for the synthesis of 1a (Perreux, et al. (2002) Tetrahedron 58: 2155-2162) was carried out following the general procedure, using 100 mg (0.752 mmol) of azide, 104 mg (0.972 mmol, 1.3 eq) of 2,6-lutidine and 207 mg (1.501 mmol, 2.0 eq) of thiobenzoic acid in chloroform (conc. 0.75 M) at reflux for 15 hours. FCC (silica gel, 20% acetone-hexane) gave 124 mg (78%) of 1a as a white solid. mp: 104-105° C. (ref 105-107° C.); Spectral data of 1a were identical with published data (Hermann, et al. (1992) supra); IR νₘₐₓ (neat)/cm⁻¹ 3321, 3080, 1641; δ_{H} (400 MHz, CDCl₃) 7.78 (2H, d, J=7.6 Hz, ArH), 7,51-7.24 (8H, series of m ArH), 6.60-6.52 (1H, bs, NH), 4.63 (2H, d, J=4.8 Hz, CH₂); δ_{C} (100 MHz, CDCl₃) 167.4, 138.2, 134.4, 131.5, 128.8 (2), 128.6 (2), 127.9 (2), 127.6, 127.0 (2), 44.1; m/z (ESIMS) 234 (M+Na)⁺.

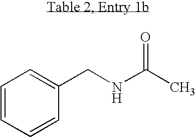

The reaction for synthesizing 1b (Agwada (1982) J. Chem. Eng. Data 27: 481-483) was carried out following the general procedure, using 237 mg (1.78 mmol) of azide, 248 mg (2.32 mmol) of 2,6-lutidine and 177 mg (2.33 mmol) of thioacetic acid in chloroform (1 M conc. of azide) at reflux for 15 hours. FCC (silica gel, 1:3 acetonehexane) gave 230 mg (86%) of 1b as a white solid. mp: 62-63° C. (ref 60-61° C.); IR νₘₐₓ (neat)/cm⁻¹ 3292, 3087, 1639; δ_{H} (400 MHz, CDCl₃) 7.31-7.21 (5H, series of m, ArH), 6.70-6.62 (1H, bs, NH), 4.32 (2H, d, J=5.6 Hz, CH₂), 1.92 (3H, s, CH₃); δ_{C} (100 MHz, CDCl₃) 170.3, 138.4, 128.6 (2), 127.7 (2), 127.3, 43.5, 23.0; m/z (ESIMS) 172 (M+Na)⁺.

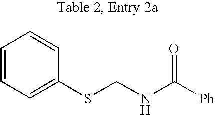

The reaction for synthesizing 2a (Vankar and Rao (1985) Tetrahedron 41: 3405-3410) was carried out following the general procedure, using 140 mg (0.848 mmol) of azide, 120 mg (1.12 mmol) of 2,6-lutidine and 152 mg (1.102 mmol) of thiobenzoic acid in chloroform (0.85 M conc. of azide) at reflux for 15 hours. FCC (silica gel, 1:8 acetone-hexane) gave 159 mg (85%) of 2a as a white solid. mp: 65-66° C. (ref 67° C.); IR νₘₐₓ (neat)/cm⁻¹ 3302, 3057, 1642; δ_{H} (400 MHz, CDCl₃) 7.68 (2H, d, J=7.6 Hz, ArH), 7,50-7.46 (3H, m ArH), 7.41 (2H, t, J=7.6 Hz, ArH), 7.32 (2H, t, J=7.2 Hz, ArH), 7.26 (1H, t, J=7.6 Hz, ArH), 6.58-6.46 (1H, bs, NH), 4.89 (2H, d, J=6.4 Hz, CH₂); δ_{C} (100 MHz, CDCl₃) 167.1, 133.9, 133.7, 131.8, 131.4 (2), 129.3 (2), 127.6 (2), 127.5, 126.9 (2), 44.3; m/z (ESIMS) 266 (M+Na)⁺.

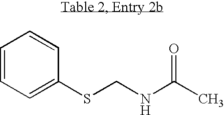

The reaction for synthesizing 2b (Vankar and Rao (1985) supra) was carried using out following the general procedure, using 157 mg (0.952 mmol) of azide, 133 mg (1.245 mmol) of 2,6-lutidine and 94 mg (1.237 mmol) of thioacetic acid in chloroform (0.95 M conc. of azide)) at reflux for 15 hours. FCC (silica gel, 1:3 acetone-hexane) gave 146 mg (85%) of 2b as a white solid. mp: 46-47° C. (ref: 45° C.); IR νₘₐₓ (neat)/cm⁻¹ 3279, 3057, 1657; δ_{H} (400 MHz, CDCl₃) 7.40 (2H, d, J=7.6 Hz, ArH), 7.29 (2H, t, J=6.8 Hz, ArH), 7.23 (1H, t, J=7.6 Hz, ArH), 6.71 (1H, bs, NH), 4.63 (2H, d, J=6.0 Hz, CH₂), 1.91 (3H, s, CH₃); δ_{C} (100 MHz, CDCl₃) 170.5, 134.4, 131.0 (2), 129.3 (2), 127.3, 44.0, 23.5; m/z (ESIMS) 204 (M+Na)⁺.

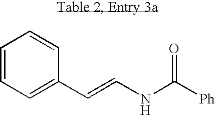

The reaction for synthesizing 3a (Shen and Porco (2000) Org. Lett. 2: 1333-1336) was carried out following the general procedure, using 50 mg (0.344 mmol) of azide, 55 mg (0.516 mmol) of 2,6-lutidine and 71 mg (0.516 mmol) of thiobenzoic acid in chloroform (conc. 1 M) at reflux for 10 hours. FCC (silica gel, 20% ethyl acetatehexane) gave 3a as a highly viscous liquid (51 mg, 66%). IR νₘₐₓ (neat)/cm⁻¹ 3262, 3297, 3053, 1657, 1636; δ_{H} (400 MHz, Acetone-d₆) 9.80-9.98 (1H, bs, NH), 8.01 (2H, d, J=7.2 Hz, ArH), 7.79 (1H, dd, J1=14.8 Hz, J2=10.2 Hz, PhCH═CH), 7.59 (1H, t, J=7.2 Hz, ArH), 7,51 (2H, dd, J1=8.0 Hz, J2=6.8 Hz, ArH), 7.40 (2H, d, J=7.6 Hz, ArH), 7,31 (2H, dd, J1=8.0 Hz, J2=7.2 Hz, ArH), 7.17 (1H, t, J=7.2, ArH), 6.46 (1H, d, J=14.8 Hz, PhCH═CH); δ_{C} (75 MHz, Acetone-d₆) 164.7, 137.6, 134.5, 132.4, 129.3 (2), 129.1 (2), 128.1 (2), 126.9 (2), 126.0, 124.6, 113.5; m/z (ESIMS) 222 (M−1)⁻.

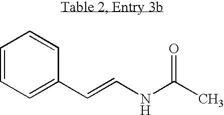

The reaction for synthesizing 3b (Alonso, et al. (1997) Tetrahedron 53: 4835-4856) was carried out following the general procedure, using 50 mg (0.344 mmol) of azide, 85 mg (0.791 mmol) of 2,6-lutidine and 60 mg (0.791 mmol) of thioacetic acid in chloroform (conc. 0.78 M) at reflux for 18 hours. FCC (silica gel, 30% ethyl acetatehexane) gave 3b as a white solid (44 mg, 79%). IR νₘₐₓ (neat)/cm⁻¹ 3262, 3191, 3054, 1660, 1639; δ_{H} (400 MHz, CDCl₃) 7.53 (1H, dd, J1=14.8 Hz, J2=10.8 Hz, PhCH═CH), 7.14-7.40 (6H, series of m, NH, ArH), 6.08 (1H, d, J=14.4 Hz, PhCH═CH), 2.11 (3H, s, CH₃); δ_{C} (75 MHz, CDCl₃) 167.1, 135.8, 128.4 (2), 126.4, 125.3 (2), 122.5, 112.2, 23.3; m/z (ESIMS) 160 (M−1)⁻.

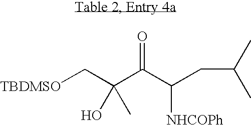

The reaction was carried out following the general procedure, using 22 mg (0.067 mmol) of azide (70:30 diastereomeric mixture), 9.3 mg (0.087 mmol) of 2,6-lutidine and 12 mg (0.087 mmol) of thiobenzoic acid in chloroform (conc. 0.33 M) at room temperature for 30 hours. FCC (silica gel, 15% ethyl acetate-hexane) gave 4a (70:30 diastereomeric mixture) as a clear viscous liquid (26 mg, 94%). When the reaction was carried with 55:45 diastereomeric mixture of azide, the corresponding amides were obtained in the same 55:45 ratio. Thus, the epimerization of the stereocenter was not observed under the reaction conditions. Spectral data for the major diastereomer: IR νₘₐₓ (neat)/cm⁻¹ 3346, 3061, 2954, 1714, 1640; δ_{H} (300 MHz, CDCl₃) 7.79 (2H, d, J=5.7 Hz, ArH), 7.51 (1H, t, J=5.4 Hz, ArH), 7.44 (2H, t, J=5.7 Hz, ArH), 6.99 (1H, d, J=6.3 Hz, NH), 5.47 (1H, t, J=6.6 Hz, CHNH), 3,96 (1H, d, J=7.2 Hz, CH₂OTBDMS), 3.69 (1H, s, OH), 3,47 (1H, d, J=7.2 Hz, CH₂OTBDMS), 1.86-1.72 (2H, m, CH₂₁Pr), 1.48-1.40 (1H, m, CH(CH₃)₂), 1.33 (3H, s, CH₃), 1.07 (3H, d, J=4.5 Hz, CH₃), 0.95 (3H, d, J=4.8 Hz, CH₃), 0.84 (9H, s, C(CH₃)₃), 0.04 (3H, s, CH₃), 0.02 (3H, s, CH₃); δ_{C} (75 MHz, CDCl₃) 214.0, 166.4, 134.0, 131.4, 128.3 (2 C), 126.8 (2 C), 80.0, 69.0, 53.2, 41.2, 25.7 (3 C), 25.1, 23.5, 21.9, 21.5, 18.1, −5.52, −5.54; m/z (ESIMS) 379 (M+).

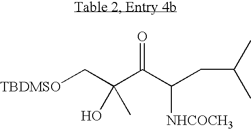

The reaction was carried out following the general procedure, using 13 mg (0.039 mmol) of azide (70:30 diastereomeric mixture), 5.5 mg (0.051 mmol) of 2,6-lutidine and 3.9 mg (0.051 mmol) of thioacetic acid in chloroform (conc. 0.18 M) at reflux for 24 hours. FCC (silica gel, 30% ethyl acetate-hexane) gave 4b (70:30 diastereomeric mixture) as a clear viscous liquid (12 mg, 88%). Spectral data for the major diastereomer: IR νₘₐₓ (neat)/cm⁻¹ 3300, 2956, 1719, 1650; δ_{H} (400 MHz, CDCl₃) 5.95 (1H, d, J=8.8 Hz, NH), 5.26 (1H, t, J=10.0 Hz, CHNH), 3,91 (1H, d, J=10.0 Hz, CH₂OTBDMS), 3.68 (1H, s, OH), 3,45 (1H, d, J=9.2 Hz, CH₂OTBDMS), 1.99 (3H, s, CH₃), 1.76-1.66 (3H, m, CH₂₁Pr, CH(CH₃)₂), 1.29 (3H, s, CH₃), 1.01 (3H, d, J=4.0 Hz, CH₃), 0.92 (3H, d, J=4.4 Hz, CH₃), 0.86 (9H, s, C(CH₃)₃), 0.05 (3H, s, CH₃), 0.04 (3H, s, CH₃); δ_{C} (75 MHz, CDCl₃) 214.0, 169.1, 79.9, 68.8, 52.5, 40.9, 25.7 (3 C), 25.0, 23.5, 23.2, 21.9, 21.5, 18.2, −5.48, −5.50; m/z (ESIMS) 318 (M+1)⁺.

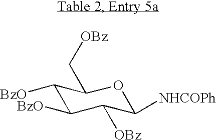

The reaction for synthesizing 5a (Avalos, et al. (1992) J. Chem. Soc. Perkin Trans. 2, 2205-2215) was carried out following the general procedure, using 144 mg (0.232 mmol) of azide (all β), 61 mg (0.570 mmol) of 2,6-lutidine and 77 mg (0.558 mmol) of thiobenzoic acid in chloroform (0.26 M conc. of azide) at reflux for 36 hours. FCC (silica gel, 1:3.5 acetone-hexane) gave 46 mg of starting material azide (all β) and 104 mg (95%, based on recovery of starting material) of 5a (all β) as a white foam. IR νₘₐₓ (neat)/cm⁻¹ 3350, 3065, 2953, 1727, 1669; δ_{H} (300 MHz, CDCl₃) 8.10-7.20 (26H, series of m, ArH), 6.14 (1H, t, J=9.6 Hz, O—CH—N), 5.83 (1H, t, J=8.7 Hz, CH—O), 5.81 (1H, t, J=9.9 Hz, CH—O), 5.56 (1H, t, J=9.6 Hz, CH—O), 4,66 (1H, dd, J1=12.3 Hz, J2=2.4 Hz, O—CH₂), 4,51 (1H, dd, J1=12.0 Hz, J2=3.9 Hz, O—CH₂), 4.40-4.36 (1H, m, CH—O); δ_{C} (75 MHz, CDCl₃) 167.42, 167.40, 166.3, 165.8, 165.3, 134.1, 133.7, 133.5, 133.3, 132.5, 130.2, 130.0, 129.9, 128.9, 128.8, 128.6, 128.5, 127.4, 79.7, 74.3, 73.2, 72.1, 69.5, 63.1.

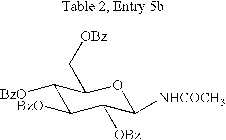

The reaction for synthesizing 5b (Sproviero (1973) Carbohydrate Research, 357-363) was carried out following the general procedure, using 116 mg (0.186 mmol) of azide (all β), 52 mg (0.486 mmol) of 2,6-lutidine and 37 mg (0.487 mmol) of thioacetic acid in chloroform (0.31 M conc. of azide) at reflux for 36 hours. FCC (silica gel, 27% acetone-hexane) gave 115 mg (97%) of 5b (all β) as a white foam. IR νₘₐₓ (neat)/cm⁻¹ 3356, 3065, 2952, 1728; δ_{H} (300 MHz, CDCl₃) 8.04 (2H, d, J=5.7 Hz), 7.96 (2H, d, J=5.7 Hz), 7.91 (2H, d, J=5.7 Hz), 7.85 (2H, d, J=5.7 Hz), 7.52-7.26 (10H, series of m), 7.20 (2H, t, J=5.7 Hz), 6.97 (1H, d, J=6.9 Hz), 6.10 (1H, t, J=7.5 Hz, CHOBz), 5.80 (1H, t, J=7.2 Hz, CHOBz), 5.73 (1H, t, J=6.9 Hz, CHOBz), 5.52 (1H, t, J=7.2 Hz, CHOBz), 4,67 (1H, dd, J1=9.0 Hz, J2=1.5 Hz, CH₂OBZ), 4,52 (1H, dd, J1=9.3 Hz, J2=3.6 Hz, CH₂OBZ), 4.37-4.34 (1H, m, CHOBZ), 1.90 (3H, s, CH₃); δ_{C} (75 MHz, CDCl₃) 170.1, 166.5, 165.9, 165.3, 164.9, 133.6, 133.3, 133.1, 132.9, 129.8, 129.6, 129.58, 129.5, 128.3, 128.2, 128.1, 78.6, 73.8, 72.9, 71.5, 69.1, 62.8, 23.3.

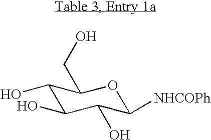

The reaction for synthesizing 1a (Sriram, et al. (1998) Acta Cryst. C 54: 1670-1672) was carried out following the general procedure, using 38 mg (0.185 mmol) of azide (all β), 62 mg (0.739 mmol) of sodium bicarbonate and 67 mg (0.486 mmol) of thiobenzoic acid (0.19 M conc. of azide) at 60° C. for 36 hours. FCC (silica gel, 1:9 methanolethyl acetate) gave 43 mg (83%) of 1a (all β) as a glassy material. IR νₘₐₓ (neat)/cm⁻¹ 3330, 3079, 2921, 1650, 1539; δ_{H} (300 MHz, CD₃OD) 7.91 (2H, t, J=7.5 Hz, ArH), 7.56 (1H, t, J=7.2 Hz, ArH), 7.47 (2H, t, J=7.8 Hz, ArH), 5.16 (1H, d, J=8.1 Hz), 4.87 (3H, s), 3.87 (1H, d, J=11.1 Hz), 3.70 (2H, dd, J1=11.7 Hz, J2=4.8 Hz, CH₂OH), 3.56-3.36 (4H, m), 2.65 (1H, s); δ_{C} (75 MHz, CD₃OD) 169.7, 134.0, 131.9, 128.3 (2 C), 127.5 (2 C), 80.6, 78.6, 77.9, 72.6, 70.3, 61.6.

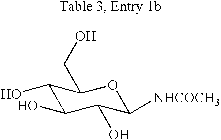

The reaction for synthesizing 1b (Sriram, et al. (1997) Acta Cryst. C 53: 1075-1077) was carried out following the general procedure, using 38 mg (0.185 mmol) of azide (all β), 62 mg (0.739 mmol) sodium bicarbonate and 36 mg (0.474 mmol) of thioacetic acid (0.19 M conc. of azide) at 60° C. for 36 hours. FCC (silica gel, 1:9 methanolacetone) gave 33 mg (80%) of 1b (all β) as a glassy material. IR νₘₐₓ (neat)/cm⁻¹ 3334, 2925, 1658; δ_{H} (400 MHz, CD₃OD) 4.94 (5H, s, OH, NH), 3,82 (1H, dd, J1=12.0 Hz, J2=1.6 Hz, CHOH), 3,64 (2H, dd, J1=12.0 Hz, J2=5.2 Hz, CH₂OH), 3.40 (1H, t, J=8.8 Hz, CHOH), 3.36-3.22 (3H, series of m), 1.99 (3H, s, CH₃); δ_{C} (75 MHz, CD₃OD) 173.1, 79.8, 78.4, 77.8, 72.8, 70.3, 61.6, 21.8.

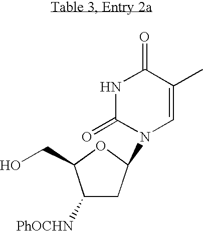

The reaction was carried out following the general procedure, using 7.7 mg (0.029 mmol) of azide, 0.7 mL of pH 7.40 buffer solution (potassium phosphate monobasic sodium hydroxide buffer, 0.05 M) and 20 mg (0.145 mmol) of thiobenzoic acid (0.04 M conc. of azide) 60° C. for 36 hours. FCC (silica gel, ethyl acetate) gave 6.7 mg (68%) of 2a as a white solid. IR νₘₐₓ (neat)/cm⁻¹ 3488, 3279, 3152, 1714, 1663, 1624; δ_{H} (300 MHz, CD₃OD) 7.93 (1H, bs, NH), 7,83 (2H, dd, J1=7.2 Hz, J2=1.2 Hz, ArH), 7.55 (1H, t, J=7.2 Hz, ArH), 7.46 (2H, t, J=7.5 Hz, ArH), 6.30 (1H, t, J=6.0 Hz), 4.88 (bs, 3H), 4.73 (1H, q, J=6.9 Hz), 4.05-3.99 (1H, m), 3,90 (1H, dd, J1=12.0 Hz, J2=2.7 Hz, CH₂OH), 3.79 (1H, dd, J1=12.0 Hz, J2=3.6 Hz, CH₂OH), 2.44 (2H, t, J=6.6 Hz, CH₂), 1.91 (3H, s, CH₃); δ_{C} (75 MHz, CD₃OD) 166.8, 164.3, 151.0, 136.9, 134.6, 132.0, 128.9 (2 C, 128.0 (2 C), 110.1, 85.7, 84.3, 62.2, 50.4, 37.8, 13.2; m/z (ESIMS) 368 (M+Na)⁺.

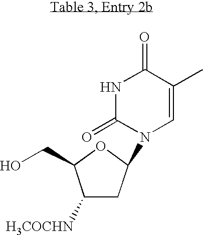

The reaction for synthesizing 2b (Hampton, et al. (1979) J. Med. Chem. 22: 621-631) was carried out following the general procedure, using 8.7 mg (0.033 mmol) of azide, 0.5 mL of pH 7.40 buffer solution (potassium phosphate monobasic sodium hydroxide buffer, 0.05 M) and 7.5 mg (0.099 mmol) of thioacetic acid (0.065 M conc. of azide) at 60° C. for 36 hours. FCC (silica gel, 1:9 methanol-ethyl acetate) gave 7.1 mg (77%) of 2b as a white solid. IR νₘₐₓ (neat)/cm⁻¹ 3488, 3338, 3266, 2955, 1685; δ_{H} (400 MHz, Acetone-d₆) 7.88 (1H, bs, NH), 7.72 (1H, d, J=5.2 Hz, NHCO), 6.21 (1H, t, J=6.0 Hz), 4.50 (1H, p, J=6.8 Hz), 3.88-3.84 (1H, m), 3.82 (1H, dd, J1=14.8 Hz, J2=2.4 Hz, CH₂OH), 3.75 (1H, dd, J1=12.0 Hz, J2=3.2 Hz, CH₂OH), 3.00-2.81 (2H, bs), 2.43-2.28 (2H, m), 1.92 (3H, s, CH₃), 1.83 (3H, s, CH₃); δ_{C} (75 MHz, CD₃OD) 171.3, 164.8, 151.7, 137.3, 111.0, 86.8, 85.0, 62.9, 50.6, 38.6, 23.3, 13.0; m/z (ESIMS) 306 (M+Na)⁺.

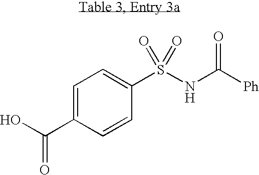

The reaction was carried out following the general procedure, using 45 mg (0.198 mmol) of azide, 37 mg (0.346 mmol) of 2,6-lutidine and 35 mg (0.254 mmol) of thiobenzoic acid (0.25 M conc. of azide) at room temperature for 1 hour. FCC (silica gel, 38:60:2 ethyl acetate-hexaneacetic acid) gave 56 mg (93%) of 3a as a white solid. mp: 253-254° C. IR νₘₐₓ (neat)/cm⁻¹ 3614, 3520, 1688; δ_{H} (400 MHz, Acetone-d₆) 8.26 (4H, s, ArH), 7.94 (2H, d, J=10.8 Hz, ArH), 7.64 (1H, t, J=9.2 Hz, ArH), 7.51 (2H, t, J=10.0 Hz, ArH); δ_{C} (100 MHz, Acetone-d₆) 166.3, 165.9, 144.5, 136.0, 134.2, 132.7, 130.9 (2), 129.6 (2), 129.4 (2), 129.1 (2); m/z (ESIMS) 304 (M−1)⁻.

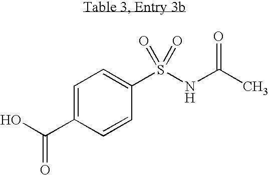

The reaction was carried out following the general procedure, using 44 mg (0.194 mmol) of azide, 37 mg (0.346 mmol) of 2,6-lutidine and 19 mg (0.250 mmol) of thioacetic acid (0.24 M conc. of azide) at room temperature for 1 hour. Removal of solvent followed by washing the residue with hexane and drying under vacuum gave 46 mg (98%) of 3b as a white solid. mp: 250° C. IR νₘₐₓ (neat)/cm⁻¹ 3541, 1692; δ_{H} (400 MHz, Acetone-d₆) 11.40-10.40 (1H, bs, NH), 8.24 (2H, d, J=8.4 Hz, ArH), 8.14 (2H, d, J=8.4 Hz, ArH), 2.04 (3H, s, CH₃); δ_{C} (75 MHz, Acetone-d₆) 168.7, 166.0, 144.1, 135.6, 130.6 (2), 128.9 (2), 23.5; m/z (ESMS) 242 (M−1)⁻.

### EXAMPLE 4

### General Procedure for Synthesizing Amides 8a-c

The thioesters 8a-c (Table 4) were prepared from 2,4,6-trimethoxybenzyl thiol (Vetter (1998) Synth. Commun. 28: 3219-3223) and the corresponding N-protected amino acid (Neises and Steglich (1978) Angew. Chem. Int. Ed. Engl. 17: 522-523). 8a was prepared from N-acetyl-Leu-OH (SIGMA). 8b and 8c were prepared from Fmoc-allo-Ile-OH (BACHEM) and Fmoc-Ile-OH (Advanced Chem Tech), respectively, in three steps: thioesterification via DCC coupling (Neises and Steglich (1978) supra), Fmoc removal, and acetylation. ¹H and ¹³C NMR indicated commercial Fmoc-allo-Ile-OH was diastereomerically pure (>95%), whereas Fmoc-Ile-OH was a diastereomeric mixture of Fmoc-Ile-OH and Fmoc-allo-Ile-OH (approximately 75:25). Conversion of the starting materials to 8b and 8c took place without measurable epimerization. Hence, 8b was obtained diastereomerically pure (>95%) and 8c was obtained as a chromatographically inseparable 75:25 mixture of diastereomers, as determined by ¹H and ¹³C NMR.

### EXAMPLE 5

### Synthesis of Exemplary 9a-e Amides

To a mixture of thioester 8a-c and triethylsilane at 0° C. was added trifluoroacetic acid-DCM (40-80% v/v) drop-wise and stirred under inert atmosphere at room temperature. After the completion of the reaction (1-3 hours), solvent was evaporated and the residue was azeotroped using benzene (5 mL). The crude thioacid was dried under vacuum and used as such for the next step.

To a solution of the above thioacid in MeOH (0.16-0.17 M conc. of thioacid) was added 2,6-lutidine and sulfonyl azide and stirred under inert atmosphere at room temperature overnight (12 hours). The solvent and the excess lutidine were evaporated. FCC (silica gel, 25% ethyl acetatehexanes and 40% acetone-hexanes buffered with 0.1% TFA) gave amide 9a-e (72-91%, two steps). No epimerisation was observed under the reaction conditions.

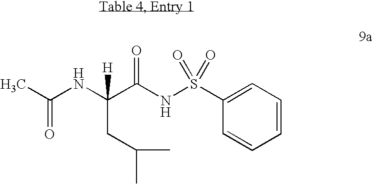

The thioester 8a (60 mg, 0.163 mmol) was deprotected (40% v/v TFA-DCM, 2 mL; Et₃SiH, 0.2 mL for 3 hours) and the resulting thioacid was converted to amide 9a (46 mg, 91%, white solid) following the general procedure, using 2,6-lutidine (52 mg, 0.49 mmol) and benzenesulfonyl azide (90 mg, 0.49 mmol) in methanol (0.16 M conc. of thioacid). mp: 201-203° C.; IR νₘₐₓ (neat)/cm⁻¹ 3333, 3039, 2864, 1708, 1640; δ_{H} (300 MHz, Acetone-d₆) 8.01 (2H, d, J=7.5 Hz, ArH), 7.72 (1H, t, J=7.5 Hz, ArH), 7.62 (2H, t, J=8.1 Hz, ArH), 7.41 (1H, d, J=5.7 Hz, NH), 4.48-4.40 (1H, m, CH), 3.30-2.70 (1H, br, NH), 1.90 (3H, s, CH₃), 1.70-1.40 (3H, m, CH, CH₂), 0.87 (3H, d, J=6.6 Hz, CH₃), 0.83 (3H, d, J=6.3 Hz, CH₃); δ_{C} (100 MHz, Acetone-d₆) 171.8, 171.0, 140.7, 134.4, 129.7 (2), 128.8 (2), 52.9, 40.5, 25.3, 23.2, 22.5, 21.8; m/z (ESIMS) 311 (M−1)⁻.

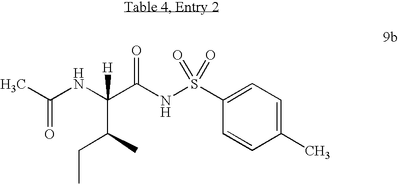

The thioester 8b (30 mg, 0.081 mmol) was deprotected (40% v/v TFA-DCM, 2 mL; Et₃SiH, 0.2 mL for 1 hour) and the resulting thioacid was converted to amide 9b (23 mg, 87%, clear viscous liquid) following the general procedure, using 2,6-lutidine (55 mg, 0.516 mmol) and p-toluenesulfonyl azide (86 mg, 0.436 mmol) in methanol (0.16 M conc. of thioacid). IR: νₘₐₓ (neat)/cm⁻¹ 3354, 3065, 2966, 1710, 1651, 1536; δ_{H} (300 MHz, Acetone-d₆) 7.89 (2H, d, J=7.8 Hz, ArH), 7.42 (2H, d, J=7.8 Hz, ArH), 7.23 (1H, d, J=7.8 Hz, NH), 4.55 (1H, dd, J=8.7, 5.1 Hz, CH), 3.68-3.40 (1H, br, NH), 2.43 (3H, s, CH₃), 1.92 (3H, s, CH₃), 1.90-1.75 (1H, m, CH), 1.36-1.25 (1H, m, CH₂), 1.22-1.08 (1H, m, CH₂), 0.84 (3H, t, J=7.5 Hz, CH₃), 0.76 (3H, d, J=6.6 Hz, CH₃); δ_{C} (75 MHz, CDCl₃) 171.8, 170.9, 145.0, 135.8, 129.5 (2), 128.3 (2), 56.8, 37.9, 25.9, 23.0, 21.7, 14.1, 11.4; m/z (ESIMS) 349 (M+Na)⁺.

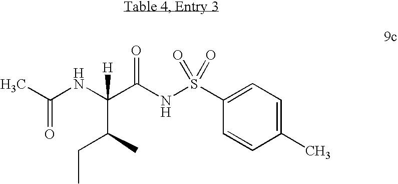

The thioester 8c (25 mg, 0.068 mmol, ˜75:25 ratio of diasteromers) was deprotected (80% v/v TFA-DCM, 2 mL; Et₃SiH, 0.2 mL for 1 hour) and the resulting thioacid was converted to amide 9c (16 mg, 72%, clear viscous liquid, in a 75:25 ratio of inseparable diastereomers) following the general procedure, using 2,6-lutidine (18.4 mg, 0.172 mmol) and p-toluenesulfonyl azide (21.5 mg, 0.109 mmol) in methanol (0.17 M conc. of thioacid). IR: νₘₐₓ (neat)/cm⁻¹ 3352, 3274, 3066, 2965, 1715, 1654, 1536; δ_{H} (300 MHz, Acetone-d₆) for major isomer: 7.89 (2H, d, J=8.4 Hz, ArH), 7.42 (2H, d, J=7.8 Hz, ArH), 7.29 (1H, d, J=8.1 Hz, NH), 4.33 (1H, t, J=7.8 Hz, CH), 3.00-2.70 (1H, br, NH), 2.43 (3H, s, CH₃), 1.92 (3H, s, CH₃), 1.86-1.70 (1H, m, CH), 1.42-1.20 (1H, m, CH₂), 1.20-1.00 (1H, m, CH₂), 0.82 (3H, d, J=6.6 Hz, CH₃), 0.79 (3H, t, J=6.9 Hz, CH₃); δ_{C} (75 MHz, CDCl₃) 171.2, 170.3, 144.7, 135.6, 129.3 (2), 128.1 (2), 57.2, 37.9, 24.6, 23.0, 21.7, 15.0, 11.0; m/z (ESIMS) 325 (M−1)⁻. The minor isomer was identical to 9b by ¹H and ¹³C.

The synthesis of 9c was also carried out under reflux conditions in chloroform solvent (7 hours, 61% yield). No epimerization (1H and ¹³C NMR) was observed under these reaction conditions.

HPLC of 9b and 9c failed to fully resolve under a variety of conditions (e.g., C-18 RP column, buffer A: 0.05% TFA in H₂O, buffer B: 0.05% TFA in CH₃CN, monitored at 220 nm. Run from 30% B to 70% B over 40 minutes. Retention time 9b: 10.83 minutes; and 9c: 10.72 minutes). NMR, however, proved reliable, providing baseline resolution of diastereomer signals in both ¹H and ¹³C NMR. Key ¹H NMR signals for 9b and 9c, which were baseline resolved in d₆-acetone and integrated for quantification, are indicated below.

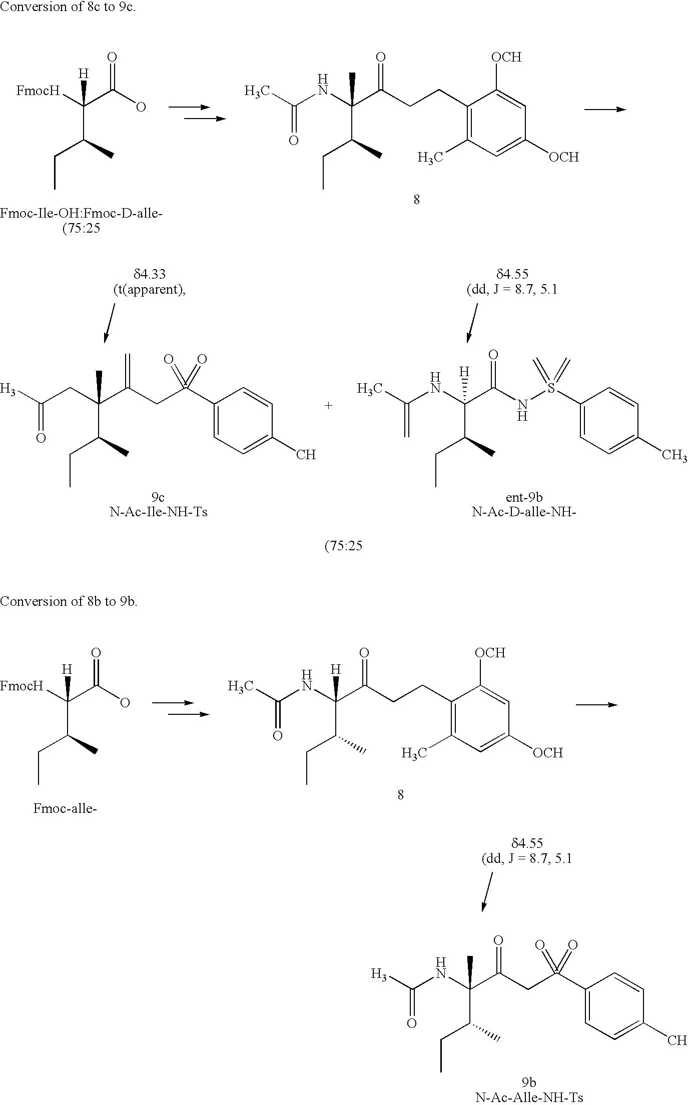

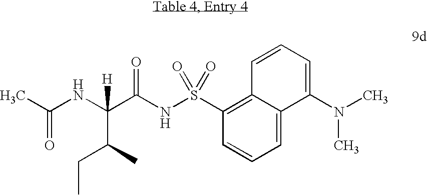

The thioester 8d (30 mg, 0.081 mmol) was deprotected (40% v/v TFA-DCM, 2 mL; Et₃SiH, 0.2 mL for 1 hour) and the resulting thioacid was converted to amide 9d (24 mg, 73%, yellow gummy liquid) following the general procedure, using 2,6-lutidine (46 mg, 0.43 mmol) and dansyl azide (45 mg, 0.162 mmol) in methanol (0.16 M conc. of thioacid). IR: νₘₐₓ (neat)/cm⁻¹ 3350, 3072, 2964, 2873, 1709, 1649, 1536; δ_{H} (300 MHz, CDCl₃) 8.59 (1H, d, J=8.4 Hz, ArH), 8.49 (1H, d, J=7.2 Hz, ArH), 8.24 (1H, d, J=8.7 Hz, ArH), 7.58 (1H, t, J=7.8 Hz, ArH), 7.50 (1H, t, J=8.4 Hz, ArH), 7.18 (1H, d, J=7.5 Hz, ArH), 6.16 (1H, d, J=9.0 Hz, NH), 4.71 (1H, dd, J=9.0, 6.6 Hz, CH), 2.87 (6H, s, 2×CH₃), 2.04 (3H, s, CH₃), 1.70-0.80 (3H, series of m, CH and CH₂), 0.66 (3H, t, J=6.9 Hz, CH₃), 0.60 (3H, d, J=6.9 Hz, CH₃); δ_{C} (75 MHz, CDCl₃) 171.3, 170.1, 151.7, 133.1, 131.9, 131.4, 129.4, 129.3, 128.3, 122.9, 118.1, 115.0, 57.0, 45.3 (2C), 37.3, 25.8, 23.0, 14.0, 11.2; m/z (ESIMS) 404 (M−1)⁻.

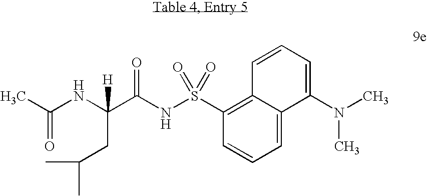

The thioester 8e (60 mg, 0.163 mmol) was deprotected (40% v/v TFA-DCM, 2 mL; Et₃SiH, 0.2 mL for 1 hour) and the resulting thioacid was converted to amide 9e (48 mg, 73%, yellow gummy liquid) following the general procedure, using 2,6-lutidine (92 mg, 0.86 mmol) and dansyl azide (90 mg, 0.326 mmol) in methanol (0.16 M conc. of thioacid). mp: 201-203° C.; IR νₘₐₓ (neat)/cm⁻¹ 3359, 3076, 2955, 2868, 1719, 1651, 1539; δ_{H} (300 MHz, CDCl₃) 8.57 (1H, d, J=7.2 Hz, ArH), 8.45 (1H, d, J=7.2 Hz, ArH), 8.24 (1H, d, J=8.7 Hz, ArH), 7.55 (1H, t, J=7.5 Hz, ArH), 7.52 (1H, t, J=7.8 Hz, ArH), 7.15 (1H, d, J=7.5 Hz, ArH), 6.11 (1H, d, J=7.5 Hz, NH), 4.62-4.54 (1H, m, CH), 2.87 (6H, s, 2×CH₃), 1.98 (3H, s, CH₃), 1.50-1.20 (3H, series of m, CH and CH₂), 0.72 (3H, d, J=6.0 Hz, CH₃), 0.66 (3H, d, J=6.0 Hz, CH₃); δ_{C} (75 MHz, CDCl₃) 171.6 (2C), 152.0, 131.8, 131.5, 129.7, 129.6, 128.5, 123.2 (2C), 118.5, 115.1, 52.2, 45.4 (2C), 40.1, 24.4, 23.0, 22.5, 22.0; m/z (ESIMS) 428 (M+Na)⁺.

1. A method for producing an amide comprising combining a thio acid and an organic azide in the presence of a solvent so that an amide is produced.
